(19) United States
(12) Patent Application Publication
(10) Pub. No.: US 2021/0215706 A1
(43) Pub. Date: Jul. 15, 2021

(54) SINGLE MOLECULE SEQUENCING IDENTIFICATION OF POST-TRANSLATIONAL MODIFICATIONS ON PROTEINS

(71) Applicant: Board of Regents, The University of Texas System, Austin, TX (US)

(72) Inventors: Edward MARCOTTE, Austin, TX (US); Eric ANSLYN, Austin, TX (US); Jagannath SWAMINATHAN, Austin, TX (US); Angela M. BARDO, Austin, TX (US); Caroline M. HINSON, Austin, TX (US); Cecil HOWARD, Austin, TX (US); Brendan FLOYD, Austin, TX (US)

(73) Assignee: Board of Regents, The University of Texas System, Austin, TX (US)

(21) Appl. No.: 17/155,298

(22) Filed: Jan. 22, 2021

Related U.S. Application Data
(63) Continuation of application No. PCT/US2019/042998, filed on Jul. 23, 2019.
(60) Provisional application No. 62/702,318, filed on Jul. 23, 2018.

(51) Int. Cl. G01N 33/68; G01N 33/58

(52) U.S. Cl. CPC G01N 33/6818; G01N 33/582; G01N 2440/10; G01N 2440/12; G01N 2440/26; G01N 2440/18; G01N 2440/30; G01N 2440/14

(57) ABSTRACT

The present disclosure provides methods of selectively label an amino acid residue on a peptide by replacing a post translational modification with a labeling moiety and sequencing the peptide to obtain the location of the amino acid residue and the identity of the post translational modification. In some aspects, the disclosure also provides methods of identifying the position, quantity, the identity of a post translational modification, or any combination thereof, in peptides which may be used for therapeutic purposes.

This application is a continuation of International Application No. PCT/US2019/042998, filed Jul. 23, 2019, which claims the benefit of priority to U.S. Provisional Application Ser. No. 62/702,318, filed on Jul. 23, 2018, the entire content of which is hereby incorporated by reference.

This invention was made with government support under Grant Nos. R35 GM122480 and OD009572 awarded by the National Institutes of Health. The government has certain rights in the invention.

### BACKGROUND

Post-translational modifications (PTMs) of proteins are covalent attachments of chemical moieties on the side chains of select amino acids or the N and C terminus of a peptide or a protein. The activity and functions of many proteins are modulated by the nature of their PTMs. Some non-limiting examples of PTMs include phosphorylation, glycosylation, alkylation, acylation, hydroxylation, or the attachment of a cofactor or nucleotide. Of the many different types of PTMs, one important class of chemical modifications—phosphorylation—is ubiquitous and extensively studied. This is due to their important role in cell-signaling and in diagnosing diseased states (Ardito et al., 2017; Stowell et al., 2015). Detecting and mapping the amino acid residues modified by PTMs is biologically important to study with its understanding translating into effective disease treatments.

One such example is the C-terminal domain of the Epidermal growth factor receptor (EGFR) family of proteins that contains approximately 20 tyrosine residues capable of being phosphorylated. Depending on the combination of these phosphorylated sites in an activated cell, the downstream processes can range from cell proliferation, differentiation, anti-apoptosis (survival), adhesion, migration, and angiogenesis (Huang et al., 2011). Understanding and mapping these sites is thus critical not only to better understand cell signaling pathways, but also develop the current therapeutic drugs. However, mapping post-translational modifications have been intrinsically challenging due to their low abundance and sample heterogeneity. The current methods do not allow for precise determination of the specific location of PTMs while also allowing for quantitative determination of the PTMs. Therefore, there remains an unmet need to identify methods which allow from improved detection of PTMs in a protein or peptide.

### SUMMARY

The present disclosure provides methods and systems for protein or peptide sequencing and/or protein or peptide identification. Methods and systems of the present disclosure may be used to sequence a protein or peptide for the determination of a post-translational modification(s) and the location(s) of such post-translational modification(s).

In some aspects, the present disclosure provides methods of identifying a post translational modification on an amino acid residue of a peptide or protein, the method comprising:
(A) treating the peptide or protein with a labeling reagent under conditions such that the labeling reagent interacts with the post translational modification on the amino acid residue of the peptide or protein, to covalently couple the labeling reagent or derivative thereof to the amino acid residue and yield a labeled peptide or protein; and
(B) sequencing the labeled peptide or protein.

In some embodiments, the post translational modification on the amino acid residue is phosphorylation, glycosylation, nitrosylation, citrullination, sulfenylation, or trimethylation. In some embodiments, the post translational modification on the amino acid residue is phosphorylation on tyrosine, serine, or threonine. In some embodiments, the post translational modification on the amino acid residue is phosphorylation on a serine. In other embodiments, the post translational modification on the amino acid residue is phosphorylation on a threonine. In other embodiments, the post translational modification on the amino acid residue is an N-glycosylation. In some embodiments, the post translational modification on the amino acid residue is glycosylation of asparagine or arginine. In other embodiments, the post translational modification on the amino acid residue is an O-glycosylation. In some embodiments, the post translational modification on the amino acid residue is glycosylation of serine, threonine, or tyrosine. In other embodiments, the post translational modification on the amino acid residue is trimethylation. In some embodiments, the post translational modification on the amino acid residue is trimethylation of lysine. In other embodiments, the post translation modification on the amino acid residue is nitrosylation. In some embodiments, the post translation modification on the amino acid residue is nitrosylation of a cysteine or tyrosine. In some embodiments, the post translation modification on the amino acid residue is nitrosylation of a cysteine. In other embodiments, the post translation modification on the amino acid residue is nitrosylation of a tyrosine. In other embodiments, the post translation modification on the amino acid residue is citrullination. In other embodiments, the post translation modification on the amino acid residue is sulfenylation. In some embodiments, the post translational modification on the amino acid residue is sulfenylation of a cysteine.

In some embodiments, the post translation modification is on an amino acid residue of a protein. In other embodiments, the post translation modification is on an amino acid residue of a peptide. In some embodiments, the labeling reagent comprises a thiol group. In some embodiments, the labeling reagent comprises two thiol groups. In some embodiments, the labeling reagent comprises an amine reactive group such as a succinimidyl ester. In some embodiments, the labeling reagent comprises a glyoxal group. In some embodiments, the labeling reagent comprises a 1,3-cycloalkanedione group such as a 1,3-hexanedione.

In some embodiments, the labeling reagent is a fluorophore, oligonucleotide, or peptide-nucleic acid. In some embodiments, the labeling reagent is a fluorophore. In some embodiments, the labeling reagent is a thiol containing fluorophore. In some embodiments, the fluorophore is a xanthene dye such as a rhodamine dye.

In some embodiments, the methods involve treating the peptide or protein with the labeling reagent comprises:
(i) reacting the peptide or protein under conditions such that the post translational modification on the peptide or protein is converted to a reactive group to form a reactive peptide or protein;
(ii) reacting the labeling reagent with the reactive peptide or protein to form the labeled peptide or protein.

In some embodiments, the reactive peptide or protein is formed by treating the peptide or protein comprising a phosphorylation post translational modification with a base. In some embodiments, the base is a rare earth metal hydroxide such as Ba(OH)₂.

In other embodiments, the reactive peptide or protein is formed by treating the peptide or protein comprising a phosphorylation post translational modification with an activating agent and a base. In some embodiments, the activating agent is a carbodiimide such as 1-ethyl-3-(3-dimethylaminopropyl)carbodiimide (EDC). In some embodiments, the base is a heteroaromatic base such as an imidazole.

In other embodiments, the reactive peptide or protein is formed by treating the peptide or protein comprising a trimethyl post translational modification with silver oxide (Ag₂O). In some embodiments, the peptide or protein comprising a trimethyl post translational modification is treated with silver oxide in the presence of heat. In some embodiments, the reactive peptide or protein is formed by treating the peptide or protein comprising a trimethyl post translational modification with a base. In some embodiments, the base is a nitrogenous base such as diisopropylethylamine or trimethylamine.

In other embodiments, the reactive peptide or protein is formed by treating the peptide or protein comprising a glycosylation post translational modification with an oxidizing agent. In some embodiments, the oxidizing agent is a hypervalent iodide reagent such as sodium periodate.

In other embodiments, the reactive peptide or protein is formed by treating the peptide or protein comprising a nitrosylation post translational modification with a reducing agent. In some embodiments, the reducing agent is disulfide reducing agent such as dithiothreitol. In some embodiments, the reducing agent further comprises heme. In some embodiments, the reactive peptide or protein is formed by treating the peptide or protein comprising a nitrosylation post translational modification with phosphine. In some embodiments, the phosphine is an unsubstituted or substituted trialkylphosphine or an unsubstituted or substituted a triarylphosphine. In some embodiments, the phosphine is an unsubstituted or substituted triarylphosphine. In some embodiments, the phosphine is an unsubstituted or substituted triphenylphosphine. In some embodiments, the methods involve contacting the peptide or protein with the labeling reagent comprises reacting the peptide or protein comprising a post translational modification with a phosphine. In some embodiments, the phosphine is an unsubstituted or substituted trialkylphosphine or an unsubstituted or substituted triarylphosphine. In some embodiments, the phosphine is an unsubstituted or substituted triarylphosphine. In some embodiments, the phosphine is an unsubstituted or substituted triphenylphosphine. In some embodiments, the phosphine is covalently linked to the labeling reagent.

In some embodiments, the methods involve contacting the peptide or protein with the labeling reagent comprises reacting the peptide or protein comprising a post translational modification with a glyoxal group. In some embodiments, the glyoxal group is covalently linked to the labeling reagent. In other embodiments, the methods involve contacting the peptide or protein with the labeling reagent comprises reacting the peptide or protein comprising a post translational modification with a 1,3-cycloalkanedione such as a 1,3-cyclohexanedione. In some embodiments, the 1,3-cycloalkanedione is covalently bonded to the labeling reagent. In some embodiments, the reactive group on the reactive peptide or protein is a double bond. In some embodiments, the reactive peptide or protein is treated with the labeling reagent comprising a thiolene-click reaction to form a labeled peptide or protein. In some embodiments, the reactive peptide or protein is treated with the labeling reagent with a double bond in the presence of an olefin metathesis reagent to form a labeled peptide or protein. In some embodiments, the reactive peptide or protein is treated with the labeling reagent comprising a cycloaddition reaction to form a labeled peptide or protein.

In some embodiments, the reactive group on the reactive peptide or protein is an aldehyde. In some embodiments, the labeling reagent is treated with the reactive group on the reactive peptide or protein comprising nucleophilic addition, nucleophilic substitution, or radical addition. In some embodiments, the labeling reagent forms a thioether when treated with the reactive group on the reactive peptide or protein. In some embodiments, the labeling reagent forms a dithiane. In some embodiments, the reactive peptide or protein is treated with the labeling reagent to form an amide bond. In some embodiments, the amide bond formation provides the labeled peptide or protein. In some embodiments, the reactive peptide or protein is treated with the labeling reagent to form a disulfide bond. In some embodiments, the disulfide bond formation provides the labeled peptide of protein. In some embodiments, the reactive peptide or protein is treated with the labeling reagent to form a heterocycloalkane. In some embodiments, the heterocycloalkyl group formation provides the labeled peptide of protein. In some embodiments, the reactive peptide or protein is treated with the labeling reagent to form a thioether bond. In some embodiments, the thioether bond formation provides the labeled peptide of protein.

In some embodiments, the sequencing comprises a fluorosequencing method. In some embodiments, the sequencing is at a single molecular level. In some embodiments, the fluorosequencing method comprises labeling at least one amino acid of the peptide or protein which does not contain a post translational modification with a second labeling reagent. In some embodiments, the fluorosequencing method comprises labeling one, two, three, four, or five distinct amino acids of the peptide or protein which do not contain a post translation modification. In some embodiments, each amino acid is labeled with a distinct second labeling reagent.

In some embodiments, the peptide or protein is bound to a solid support such as a surface. In some embodiments, the solid support is a resin, a bead, or a modified glass surface. In some embodiments, the solid support is the modified glass surface such as an aminosilicate surface.

In some embodiments, the fluorosequencing method further comprises removing at least one amino acid residue of the peptide or protein. In some embodiments, the fluorosequencing method comprises sequentially removing two or more consecutive amino acid residues of the peptide or protein. In some embodiments, the fluorosequencing method comprises sequentially removing amino acid residues of the peptide or protein until a labeled amino acid comprising a modified post translational modification is removed. In some embodiments, the fluorosequencing method comprises sequentially removing from 1 to 20 amino acid residues of the peptide or protein until a labeled amino acid comprising a modified post translational modification is removed. In some embodiments, the amino acid residues are removed by Edman degradation. In some embodiments, the amino acid residue is removed by treating the N-terminal amino acid residue with a thiourea and an acid, microwave irradiation, or heat. In some embodiments, the amino acid residues are removed by an enzyme.

In some embodiments, the peptide or protein is digested by a protease. In some embodiments, the peptide or protein is digested by a protease before labeling the amino acid comprising the post translational modification. In some embodiments, the peptide or protein is obtained from a biological sample. In some embodiments, the biological sample is a cell-free biological sample. In some embodiments, the biological sample is derived from blood. In other embodiments, the biological sample is derived from urine. In other embodiments, the biological sample is derived from mucous. In other embodiments, the biological sample is derived from saliva.

In some embodiments, a covalent bond between the post translational modification on the amino acid residue of the peptide or protein and the labeling reagent is formed. In some embodiments, the labeling reagent or derivative thereof is directly covalently bonded to the amino acid residue. In some embodiments, the labeling reagent or derivative thereof is covalently coupled to the amino acid residue through an intermediary molecule.

In still another aspect, the present disclosure provides methods of determining the status of a disease or disorder in a subject, the method comprising:
(A) detecting a change in a type, identity, quantity, or position of a post translational modification or a plurality of post translational modifications on a protein or peptide using the methods described herein; and
(B) determining the status of the disease or disorder in the subject according to at least said change.

In some embodiments, the methods further comprise obtaining a biological sample from the subject. In some embodiments, determining the status of a disease or disorder is determining the prognosis of the patient that has the disease. In other embodiments, determining the status of a disease or disorder is diagnosing the patient with the disease. In other embodiments, determining the status of a disease or disorder is determining if the patient is at risk of having the disease.

In some embodiments, the change in post translation modification of a protein or peptide is a change in the phosphorylation of the protein. In other embodiments, the change in post translation modification of a protein or peptide is a change in the trimethylation of the protein. In other embodiments, the change in post translation modification of a protein or peptide is a change in the glycosylation of the protein. In other embodiments, the change in post translation modification of a protein or peptide is a change in the nitrosylation of the protein. In some embodiments, the change in post translation modification of a protein or peptide is a change in the citrullination of the protein. In some embodiments, the change in post translation modification of a protein or peptide is a change in the sulfenylation of the protein.

In some embodiments, the biological sample is a cell-free biological sample such as saliva, mucous, urine, serum, plasma, or whole blood. In some embodiments, the method conveys the presence of one or more post translational modifications. In some embodiments, the method conveys the presence of two or more post translation modifications. In some embodiments, the method conveys the absence of one or more post translational modifications. In some embodiments, the method conveys the absence of one or more post translational modifications and the presence of one or more post translational modifications.

In some embodiments, the method conveys the type of the post translational modification in the protein. In some embodiments, the method conveys the identity of the post translational modification in the protein. In some embodiments, the method conveys the quantity of the post translational modification in the protein. In some embodiments, the method conveys the position of the post translational modification in the protein. In some embodiments, the subject is a mammal such as a human.

In some embodiments, the method further comprises enriching the protein before determining the type, identity, quantity, or position of the post translational modifications. In some embodiments, the protein is enriched by purification of the biological sample. In some embodiments, the protein is subjected to degradation before determining the types or identities of the post translational modifications. In some embodiments, the protein is degraded by a protease.

In some embodiments, the protein is immobilized on a solid support. In some embodiments, the solid support is a surface. In some embodiments, the solid support is a resin, a bead, or a modified glass surface. In some embodiments, the solid support is the modified glass surface such as an aminosilicate surface.

In some embodiments, the method comprises determining the type, identity, quantity, or position of post translational modification on two or more peptides or proteins.

In yet another aspect, the present disclosure provides methods for determining the status of a disease or disorder in a subject, the method comprising:
detecting a change in a type, identity, quantity, or position of the post translational modifications on the protein or peptide using the methods described herein related to the disease or disorder.

In some embodiments, the methods further comprise obtaining a biological sample from the subject.

In still another aspect, the present disclosure provides modified peptides or proteins comprising a peptide or protein comprising one or more post translational modifications, wherein at least one post translational modification of said peptide or protein comprising one or more post translational modifications is altered with at least a first labeling moiety, thereby forming a labeled peptide or protein comprising one or more post translational modifications.

In some embodiments, the at least the first labeling moiety is a fluorophore. In some embodiments, the peptide or protein comprises a second labeling moiety attached to one or more amino acid residues of the peptide or protein. In some embodiments, the second labeling moiety is a fluorophore. In some embodiments, said at least one post translational modification is selected from the group consisting of phosphorylation, glycosylation, nitrosylation, citrullination, sulfenylation, trimethylation, or any combination thereof. In some embodiments, each post translational modification selected from the group consisting of phosphorylation, glycosylation, nitrosylation, citrullination, sulfenylation, or trimethylation is altered by a distinct labeling moiety. In some embodiments, the modified peptide or protein comprises from 3 amino acid residues to about 250 amino acid residues. In some embodiments, the modified peptide or protein comprises from 5 amino acid residues to about 100 amino acid residues. In some embodiments, the modified peptide or protein comprises from about 7 amino acid residues to about 50 amino acid residues.

In some embodiments, the first labeling reagent replaces the post translational modification on the amino acid residue. In some embodiments, the post translation modification is on an amino acid residue of a protein. In other embodiments, the post translation modification is on an amino acid residue of a peptide. In some embodiments, the first labeling reagent comprises a thiol group. In some embodiments, the first labeling reagent comprises two thiol groups. In some embodiments, the first labeling reagent comprises an amine reactive group such as a succinimidyl ester. In some embodiments, the first labeling reagent comprises a glyoxal group. In some embodiments, the first labeling reagent comprises a 1,3-cycloalkanedione group such as a 1,3-hexanedione.

In some embodiments, the first or second labeling reagent are a fluorophore, oligonucleotide, or peptide-nucleic acid. In some embodiments, the one of the first or second labeling reagent is a fluorophore. In some embodiments, the labeling reagent is a thiol containing fluorophore. In some embodiments, the fluorophore is a xanthene dye such as a rhodamine dye.

In some embodiments, the second labeling moiety is attached to a different type of amino acid of the peptide or protein than the first labeling moiety. In some embodiments, the methods further comprise one or more additional labeling moieties attached to one or more distinct amino acids of the peptide or protein.

In some embodiments, the peptide or protein is immobilized adjacent to a solid support. In some embodiments, the solid support is a surface. In some embodiments, the solid support is a resin, a bead, or a modified glass surface. In some embodiments, the solid support is a modified glass surface such as an aminosilicate surface.

In some embodiments, the peptide or protein has been degraded by a protease. In some embodiments, the post translation modification is phosphorylation of the peptide or protein. In other embodiments, the post translation modification is trimethylation of the peptide or protein. In other embodiments, the post translation modification is glycosylation of the peptide or protein. In other embodiments, the post translation modification is nitrosylation of the peptide or protein. In other embodiments, the post translation modification is citrullination of the peptide or protein. In other embodiments, the post translation modification is sulfenylation of the peptide or protein.

In some embodiments, the post translational modification on the amino acid residue is phosphorylation on tyrosine, serine, or threonine. In some embodiments, the post translational modification on the amino acid residue is phosphorylation on a serine. In other embodiments, the post translational modification on the amino acid residue is phosphorylation on a threonine. In other embodiments, the post translational modification on the amino acid residue is an N-glycosylation. In some embodiments, the post translational modification on the amino acid residue is glycosylation of asparagine or arginine. In other embodiments, the post translational modification on the amino acid residue is an O-glycosylation. In some embodiments, the post translational modification on the amino acid residue is glycosylation of serine, threonine, or tyrosine. In other embodiments, the post translational modification on the amino acid residue is trimethylation. In some embodiments, the post translational modification on the amino acid residue is trimethylation of lysine. In other embodiments, the post translation modification on the amino acid residue is nitrosylation. In some embodiments, the post translation modification on the amino acid residue is nitrosylation of a cysteine or tyrosine. In some embodiments, the post translation modification on the amino acid residue is nitrosylation of a cysteine. In other embodiments, the post translation modification on the amino acid residue is nitrosylation of a tyrosine. In other embodiments, the post translation modification on the amino acid residue is citrullination. In other embodiments, the post translation modification on the amino acid residue is sulfenylation. In some embodiments, the post translational modification on the amino acid residue is sulfenylation of a cysteine.

In another aspect, the present disclosure provides methods of sequencing a peptide or protein comprising:
(A) obtaining a cell-free biological sample and separating the peptide or protein from the cell-free biological sample;
(B) labeling the peptide or protein under conditions sufficient to interact with at least one amino acid residue of the peptide or protein associated with a post translational modification with a first labeling moiety to form at least one labeled amino acid residue of the peptide or protein;
(C) subjecting the peptide or protein to conditions sufficient to remove one or more individual amino acid residues from the peptide or protein; and
(D) detecting at least one signal from the at least one labeled amino acid residue, thereby identifying the sequence of the peptide or protein.

In some embodiments, the post translational modification on the amino acid residue is phosphorylation, glycosylation, nitrosylation, citrullination, sulfenylation, or trimethylation. In some embodiments, the post translational modification on the amino acid residue is phosphorylation on tyrosine, serine, or threonine. In some embodiments, the post translational modification on the amino acid residue is phosphorylation on a serine. In other embodiments, the post translational modification on the amino acid residue is phosphorylation on a threonine. In other embodiments, the post translational modification on the amino acid residue is an N-glycosylation. In some embodiments, the post translational modification on the amino acid residue is glycosylation of asparagine or arginine. In other embodiments, the post translational modification on the amino acid residue is an O-glycosylation. In some embodiments, the post translational modification on the amino acid residue is glycosylation of serine, threonine, or tyrosine. In other embodiments, the post translational modification on the amino acid residue is trimethylation. In some embodiments, the post translational modification on the amino acid residue is trimethylation of lysine. In other embodiments, the post translation modification on the amino acid residue is nitrosylation. In some embodiments, the post translation modification on the amino acid residue is nitrosylation of a cysteine or tyrosine. In some embodiments, the post translation modification on the amino acid residue is nitrosylation of a cysteine. In other embodiments, the post translation modification on the amino acid residue is nitrosylation of a tyrosine. In other embodiments, the post translation modification on the amino acid residue is citrullination. In other embodiments, the post translation modification on the amino acid residue is sulfenylation. In some embodiments, the post translational modification on the amino acid residue is sulfenylation of a cysteine.

In some embodiments, the labeling reagent replaces the post translational modification on the amino acid residue. In some embodiments, the post translation modification is on an amino acid residue of a protein. In other embodiments, the post translation modification is on an amino acid residue of a peptide. In some embodiments, the labeling reagent comprises a thiol group. In some embodiments, the labeling reagent comprises two thiol groups. In some embodiments, the labeling reagent comprises an amine reactive group such as a succinimidyl ester. In some embodiments, the labeling reagent comprises a glyoxal group. In some embodiments, the labeling reagent comprises a 1,3-cycloalkanedione group such as a 1,3-hexanedione.

In some embodiments, the labeling reagent is a fluorophore, oligonucleotide, or peptide-nucleic acid. In some embodiments, the labeling reagent is a fluorophore. In some embodiments, the labeling reagent is a thiol containing fluorophore. In some embodiments, the fluorophore is a xanthene dye such as a rhodamine dye.

In some embodiments, the methods further comprise labeling the peptide or protein with the first labeling moiety comprises:
(i) treating the peptide or protein under conditions such that the post translational modification on the peptide or protein is converted to a reactive group to form a reactive peptide or protein;
(ii) treating the first labeling moiety with the reactive peptide or protein to form a labeled peptide or protein.

In some embodiments, the reactive peptide or protein is formed by treating the peptide or protein comprising a phosphorylation post translational modification with a base. In some embodiments, the base is a rare earth metal hydroxide such as Ba(OH)₂.

In other embodiments, the reactive peptide or protein is formed by treating the peptide or protein comprising a phosphorylation post translational modification with an activating agent and a base. In some embodiments, the activating agent is a carbodiimide such as 1-ethyl-3-(3-dimethylaminopropyl)carbodiimide (EDC). In some embodiments, the base is a heteroaromatic base such as an imidazole.

In other embodiments, the reactive peptide or protein is formed by treating the peptide or protein comprising a trimethyl post translational modification with silver oxide (Ag₂O). In some embodiments, the peptide or protein comprising a trimethyl post translational modification is treated with silver oxide in the presence of heat. In some embodiments, the reactive peptide or protein is formed by treating the peptide or protein comprising a trimethyl post translational modification with a base. In some embodiments, the base is a nitrogenous base such as diisopropylethylamine or trimethylamine.

In other embodiments, the reactive peptide or protein is formed by treating the peptide or protein comprising a glycosylation post translational modification with an oxidizing agent. In some embodiments, the oxidizing agent is a hypervalent iodide reagent such as sodium periodate.

In other embodiments, the reactive peptide or protein is formed by treating the peptide or protein comprising a nitrosylation post translational modification with a reducing agent. In some embodiments, the reducing agent is disulfide reducing agent such as dithiothreitol. In some embodiments, the reducing agent further comprises heme. In some embodiments, the reactive peptide or protein is formed by treating the peptide or protein comprising a nitrosylation post translational modification with phosphine. In some embodiments, the phosphine is an unsubstituted or substituted trialkylphosphine or an unsubstituted or substituted a triarylphosphine. In some embodiments, the phosphine is an unsubstituted or substituted triarylphosphine. In some embodiments, the phosphine is an unsubstituted or substituted triphenylphosphine. In some embodiments, the methods involve contacting the peptide or protein with the labeling reagent comprises reacting the peptide or protein comprising a post translational modification with a phosphine. In some embodiments, the phosphine is an unsubstituted or substituted trialkylphosphine or an unsubstituted or substituted triarylphosphine. In some embodiments, the phosphine is an unsubstituted or substituted triarylphosphine. In some embodiments, the phosphine is an unsubstituted or substituted triphenylphosphine. In some embodiments, the phosphine is covalently linked to the labeling reagent.

In some embodiments, the methods involve contacting the peptide or protein with the labeling reagent comprises reacting the peptide or protein comprising a post translational modification with a glyoxal group. In some embodiments, the glyoxal group is covalently linked to the labeling reagent. In other embodiments, the methods involve contacting the peptide or protein with the labeling reagent comprises reacting the peptide or protein comprising a post translational modification with a 1,3-cycloalkanedione such as a 1,3-cyclohexanedione. In some embodiments, the 1,3-cycloalkanedione is covalently bonded to the labeling reagent. In some embodiments, the reactive group on the reactive peptide or protein is a double bond. In some embodiments, the reactive peptide or protein is treated with the labeling reagent comprising a thiolene-click reaction to form a labeled peptide or protein. In some embodiments, the reactive peptide or protein is treated with the labeling reagent with a double bond in the presence of an olefin metathesis reagent to form a labeled peptide or protein. In some embodiments, the reactive peptide or protein is treated with the labeling reagent comprising a cycloaddition reaction to form a labeled peptide or protein.

In some embodiments, the reactive group on the reactive peptide or protein is an aldehyde. In some embodiments, the labeling reagent is treated with the reactive group on the reactive peptide or protein comprising nucleophilic addition, nucleophilic substitution, or radical addition. In some embodiments, the labeling reagent forms a thioether when treated with the reactive group on the reactive peptide or protein. In some embodiments, the labeling reagent forms a dithiane. In some embodiments, the reactive peptide or protein is treated with the labeling reagent to form an amide bond. In some embodiments, the amide bond formation provides the labeled peptide or protein. In some embodiments, the reactive peptide or protein is treated with the labeling reagent to form a disulfide bond. In some embodiments, the disulfide bond formation provides the labeled peptide of protein. In some embodiments, the reactive peptide or protein is treated with the labeling reagent to form a heterocycloalkane. In some embodiments, the heterocycloalkyl group formation provides the labeled peptide of protein. In some embodiments, the reactive peptide or protein is treated with the labeling reagent to form a thioether bond. In some embodiments, the thioether bond formation provides the labeled peptide of protein.

In some embodiments, the sequencing comprises a fluorosequencing method. In some embodiments, the sequencing is at a single molecular level. In some embodiments, the fluorosequencing method comprises labeling at least one amino acid of the peptide or protein which does not contain a post translational modification with a second labeling reagent. In some embodiments, the fluorosequencing method comprises labeling one, two, three, four, or five distinct amino acids of the peptide or protein which do not contain a post translation modification. In some embodiments, each amino acid is labeled with a distinct second labeling reagent.

In some embodiments, the peptide or protein is bound to a solid support such as a surface. In some embodiments, the solid support is a resin, a bead, or a modified glass surface. In some embodiments, the solid support is the modified glass surface such as an aminosilicate surface.

In some embodiments, the fluorosequencing method further comprises removing at least one amino acid residue of the peptide or protein. In some embodiments, the fluorosequencing method comprises sequentially removing two or more consecutive amino acid residues of the peptide or protein. In some embodiments, the fluorosequencing method comprises sequentially removing amino acid residues of the peptide or protein until a labeled amino acid comprising a modified post translational modification is removed. In some embodiments, the fluorosequencing method comprises sequentially removing from 1 to 20 amino acid residues of the peptide or protein until a labeled amino acid comprising a modified post translational modification is removed. In some embodiments, the amino acid residues are removed by Edman degradation. In some embodiments, the amino acid residue is removed by treating the N-terminal amino acid residue with a thiourea and an acid, microwave irradiation, or heat. In some embodiments, the amino acid residues are removed by an enzyme.

In some embodiments, the peptide or protein is digested by a protease. In some embodiments, the peptide or protein is digested by a protease before labeling the amino acid comprising the post translational modification.

In yet another aspect, the present disclosure provides methods for polypeptide sequence identification, comprising:
(A) obtaining a first polypeptide from a cell-free biological sample of a subject;
(B) using said first polypeptide to generate a second polypeptide immobilized to a support, wherein said second polypeptide comprises labeled amino acids;
(C) subjecting said second polypeptide to conditions sufficient to remove amino acids from said polypeptide; and
(D) during or subsequent to removal of said amino acids from said polypeptide, detecting signals from at least a subset of said labeled amino acids, thereby identifying a sequence of said second polypeptide to determine a sequence of said first polypeptide from said cell-free biological sample.

In some embodiment, less than all types of amino acids of said second polypeptide are labeled. In some embodiments, said first polypeptide is a protein.

In still yet another aspect, the present disclosure provides methods for processing or analyzing a protein or peptide containing or suspected of containing at least one post-translational modification, comprising:
(A) sequencing said protein or peptide, and
(B) identifying said at least one post-translational modification in at least one amino acid subunit of said protein or peptide, or derivative thereof.

In some embodiments, said sequencing comprises subjecting said protein or peptide to degradation conditions to sequentially remove amino acid sub-units from said protein or peptide, and detecting at least a subset of said amino acid sub-units. In some embodiments, less than all amino acid sub-units of said peptide or protein are labeled, and wherein said sequencing comprises detecting a subset of said amino acid sub-units. In some embodiments, said at least one post-translational modification is identified during said sequencing. In some embodiments, said at least one post-translational modification is identified prior to said sequencing. In some embodiments, said protein or peptide is obtained from a sample and processed to label said at least one post-translational modification. In some embodiments, said sample is a cell-free sample. In some embodiments, said sequencing comprises labeling said at least one post-translational modification of said protein or peptide with a label, and detecting said label to thereby identify said at least one post-translational modification on said protein or peptide.

In yet another aspect, the present disclosure provides methods for processing or analyzing a protein or peptide, comprising subjecting said protein or peptide to conditions sufficient to specifically label different post-translational modifications of said protein or peptide, and detecting labels corresponding to said different post-translational modifications of said protein or peptide to thereby detect said different post-translational modifications of said protein or peptide.

In some embodiments, said different post-translational modifications comprise phosphorylation, glycosylation, nitrosylation, citrullination, sulfenylation, or trimethylation.

As used herein, “essentially free,” in terms of a specified component, may refer to a specified component being absent from a composition or the component is present as a contaminant or in trace amounts. The total amount of the specified component resulting from any unintended contamination of a composition can be below 0.1%. In some embodiments, a composition in which no amount of the specified component can be detected with standard analytical methods.

As used herein in the specification and claims, “a” or “an” may refer to one or more. As used herein in the specification and claims, when used in conjunction with the word “comprising”, the words “a” or “an” may refer to one or more than one. As used herein, in the specification and claim, “another” or “a further” may refer to at least a second or more.

As used herein in the specification and claims, the term “about” is used to indicate that a value includes the inherent variation of error for the device, the method being employed to determine the value, or the variation that exists among the study subjects. In some embodiments, the term “about” refers to ±5% of the listed value.

Other objects, features and advantages of the present disclosure will become apparent from the following detailed description. The detailed description and the specific examples, while indicating certain embodiments, are given by way of illustration, since various changes and modifications within the spirit and scope will become apparent from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Correct identification of phosphoserine residues on synthetic CTD heptad peptide by fluorosequencing. (Top) Phosphoserine is present at the 2^{nd} position. (Bottom) Phosphoserine is present at the 5^{th} position. Representative raw imaging data are shown for two individual peptide molecules from each experiment. For each individual molecule, the images are organized as a horizontal strip of consecutive ‘FIRE’ micrographs (each corresponding to a square of 3×3 microns) centered on the peptide molecule. Each image represents one successive observation of emitted fluorescent light from that molecule after a round of Edman chemistry. A sharp reduction in fluorescence follows the Edman cycle in which the amino acid with the attached fluorescent dye was removed, thus revealing the amino acid sequence position of the phosphorylated residue in the original peptide. The heatmap denotes the frequency histogram, tallying the counts of individual peptide molecules having lost fluorescence after every Edman degradation cycle over the background counts. The phosphorylated serine residue in the 2^{nd} position (top) and 5^{th} position (bottom) have significantly higher counts of fluorescent loss at the 2^{nd} and 5^{th} position, respectively, when analyzed by the fluorosequencing method.

FIG. 2 shows fluorosequencing position counts between two biological samples. Proteins from two different HEK-293T samples were digested, labeled, and sequenced on the fluorosequencing platform. Read counts were observed to be highly correlated between these biological replicates (Pearson coefficient 0.9582). Data is counts and plotted on a log 10 scale

### DESCRIPTION OF ILLUSTRATIVE EMBODIMENTS

In some aspects, the present disclosure provides methods of typing, identifying, quantifying, or locating a post translational modification (PTM) in a peptide or protein. These methods may be used to determine the type, location, quantity, or position of a PTM such as phosphorylation, glycosylation, or alkylation in a peptide or protein. These methods may be used in conjunction with a fluorosequencing method such as those which include labeling of the post translational modification with a labeling moiety such as a fluorophore. These methods may further include the removal of one or more amino acid residues from the peptide or protein. In some aspects, these methods may be used to determine the progression or status of a disease or disorder in a patient.

### I. PEPTIDE SEQUENCING METHODS

There exist many methods of identifying the sequence of a peptide including fluorosequencing, mass spectroscopy, identifying the peptide sequence from the nucleic acid sequence, and Edman degradation. Fluorosequencing has been found to provide single molecule resolution for the sequencing of proteins of interest (Swaminathan, 2010; U.S. Pat. No. 9,625,469; U.S. patent application Ser. No. 15/461,034; U.S. patent application Ser. No. 15/510,962). One of the hallmarks of fluorosequencing is introduction of a fluorophore or other label into specific amino acid residues of the peptide sequence. This can involve the introduction of one or more amino acid residues with a unique labeling moiety. In some embodiments, one, two, three, four, five, or more different amino acids residues are labeled with a labeling moiety. The labeling moiety that may be used include fluorophores, chromophores, or a quencher. Each of these amino acid residues may include cysteine, lysine, glutamic acid, aspartic acid, tryptophan, tyrosine, serine, threonine, arginine, histidine, methionine, asparagine, and glutamine. Each of these amino acid residues may be labeled with a different labeling moiety. In some embodiments, multiple amino acid residues may be labeled with the same labeling moiety such as aspartic acid and glutamic acid or asparagine and glutamine. While this technique may be used with labeling moieties such as those described above, it is also contemplated that other labeling moiety may be used in fluorosequencing-like methods such as synthetic oligonucleotides or peptide-nucleic acid may be used. In particular, the labeling moiety used in the instant applications may be suitable to withstand the conditions of removing one or more of the amino acid residues. Some non-limiting examples of potential labeling moieties that may be used in the instant methods include those which emit a fluorescence signal in the red to infrared spectra such as an Alexa Fluor® dye, an Atto dye, a rhodamine dye, or other similar dyes. Examples of each of these dyes which were capable of withstanding the conditions of removing the amino acid residues include Alexa Fluor® 405, Rhodamine B, tetramethyl rhodamine, Alexa Fluor 555, Atto647N, and (5)6-napthofluorescein. In other aspects, it is contemplated that the labeling moiety may be a fluorescent peptide or protein or a quantum dot.

Alternatively, synthetic oligonucleotides or oligonucleotide derivatives may be used as the labeling moiety for the peptides. For example, thiolated oligonucleotides may be coupled to peptides using the presented methods. Commonly available thiol modifications are 5′ thiol modifications, 3′ thiol modifications, and dithiol modifications and each of these modifications may be used to modify the peptide. Following oligonucleotide coupling to the peptides as above, the peptides may be subjected to Edman degradation (Edman et al., 1950) and the oligonucleotides may be used to determine the presence of a specific amino acid residue in the remaining peptide sequence. In other embodiments, the labeling moiety may be a peptide-nucleic acid. The peptide-nucleic acid may be attached to the peptide sequence on specific amino acid residues.

One element of fluorosequencing is the removal of the labeled peptides through such techniques such as Edman degradation and subsequent visualization to detect a reduction in fluorescence, indicating a specific amino acid has been cleaved. Removal of each amino acid residue is carried out through a variety of different techniques including Edman degradation and proteolytic cleavage. In some embodiments, the techniques include using Edman degradation to remove the terminal amino acid residue. In other embodiments, the techniques involve using an enzyme to remove the terminal amino acid residue. These terminal amino acid residues may be removed from either the C terminus or the N terminus of the peptide chain. In situations in which Edman degradation is used, the amino acid residue at the N terminus of the peptide chain is removed.

In some aspects, the methods of sequencing or imaging the peptide sequence may comprise immobilizing the peptide on a surface. The peptide may be immobilized using an cysteine residue, the N terminus, or the C terminus. In some embodiments, the peptide is immobilized by reacting the cysteine residue with the surface. In some embodiments, the present disclosure contemplates immobilizing the peptides on a surface such as a surface that is optically transparent across the visible spectra, the infrared spectra, or a combination thereof possesses a refractive index between 1.3 and 1.6, is between 10 to 50 nm thick, is chemically resistant to organic solvents as well as strong acid such as trifluoroacetic acid, or any combination thereof. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluorous alkanes etc) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein. In other embodiments, the methods described herein may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. In other embodiments, the surface is amine functionalized. In other embodiments, the surface is thiol functionalized.

Each of these sequencing techniques involves imaging the peptide sequence to determine the presence of one or more labeling moiety on the peptide sequence. In some embodiments, these images are taken after each removal of an amino acid residue and used to determine the location of the specific amino acid in the peptide sequence. In some embodiments, the methods can result in the elucidation of the location of the specific amino acid in the peptide sequence. These methods may be used to determine the locations of specific amino acid residues in the peptide sequence or these results may be used to determine the entire list of amino acid residues in the peptide sequence. The methods may involve determining the location of one or more amino acid residues in the peptide sequence and comparing these locations to specific peptide sequences and determining the entire list of amino acid residues in the peptide sequence.

In some aspects, the methods may comprise labeling one or more additional amino acid residues which do not contain a post translational modification. These amino acids may be labeled with a labeling moiety which is different from the label used to label the amino acid residue containing the post translational modification. If more than one position on the peptide is labeled, it is contemplated that the amino acids are labeled in the following order: cysteine, lysine, N terminus, C terminus, amino acids with carboxylic acid groups on the side chain, tryptophan, or any combination thereof. It is contemplated that one or more of these particular amino acids may be labeled or all of these amino acid residues may be labeled with different labels.

In some aspects, the imaging methods used in the sequencing techniques may involve a variety of different methods such as fluorimetry and fluorescence microscopy. The fluorescent methods may employ such fluorescent techniques such as fluorescence polarization, Forster resonance energy transfer (FRET), or time-resolved fluorescence. In some embodiments, fluorescence microscopy may be used to determine the presence of one or more fluorophores in the single molecule quantity. Such imaging methods may be used to determine the presence or absence of a label on a specific peptide sequence. After repeated cycles of removing an amino acid residue and imaging the peptide sequence, the position of the labeled amino acid residue can be determined in the peptide.

### II. POST TRANSLATIONAL MODIFICATIONS

In some aspects, the present methods comprise labeling and determining the presence and position, location, quantity, type of a post translational modification of a peptide sequence, or any combination thereof. Post translational modifications are used to refer to a covalent modification of a protein or peptide through enzymatic or non-enzymatic modification of the protein or peptide. As used herein, the post translational modification includes both natural as well as non-natural modifications. Post translational modifications may be used to describe a variety of different types of covalent modifications including a modification to the side chain of an amino acid or cleaving of peptide (or amide) bonds, or as a result of oxidative stress. Often post translational modifications are attached to the side chain of an amino acid. These side chains of amino acids which contain a nucleophilic side chain are often the site of a post translational modification. The side chains of amino acids, which may be modified, include nucleophilic sites such as the hydroxyl groups of amino acids serine, threonine, and tyrosine, the amine group of amino acids lysine, arginine, and histidine, the thiol group of cysteine, and the carboxylic acid group of aspartate and glutamine.

Some non-limiting examples of post translational modifications include addition of a hydrophobic group such as alkylation which may be used to introduce one or more alkyl such as methyl groups, acylation which may be used to introduce one or more acyl group such as acetylation, formylation, or acylation with a fatty acid, or prenylation which introduces a isoprenoid group. Other post translational modifications may include the introduction of a cofactor or translation factors such as a flavin moiety, a heme moiety, lipoylation, or diphthamide formation. Other post translation modification may comprise the introduction of another protein such as SUMOylation, which attaches a SUMO protein, or ubiquitination, which attaches the protein ubiquitin.

Post translational modifications may further comprise the introduction of a chemical group to an existing amino acid residue. Some non-limiting examples of chemical groups which can be used to modify an amino acid residue include acylation, alkylation, amide bond formulation, carboxylation, glycosylation, hydroxylation, iodination, phosphorylation, nitrosylation, sulfinylation, sulfenylation, sulfation, or succinylation. In some embodiments, the present methods may be used to determine the presence of one or more of these post translational modifications. In some embodiments, the post translational modification is an alkylation specifically a methylation to introduce a mono, di or trimethylamine group to the side chain of the lysine residue. In other embodiments, the post translational modification is the phosphorylation of a hydroxyl group on tyrosine, threonine, or serine residue especially a threonine or a serine residue. In still another embodiment, the post translational modification is a glycosylation of a nitrogen or oxygen atom in the side chain of an amino acid.

The peptides or proteins with a post translational modification described herein may be obtained from a biological sample. These biological samples may be obtained from an animal or plant source. One potential animal source is a mammal source such as a sample obtained from a human. The human source may be obtained from a baby, an adolescent, or an adult human. These biological samples may include cell-free samples. A cell-free sample may be a sample which is free of cells, substantially free of cells or essentially free of cells. A cell-free biological sample may include a protein(s), peptide(s), amino acid(s), a nucleic acid molecule(s) (e.g., ribonucleic acid molecule or deoxyribonucleic acid molecule), or any combination thereof. While a sample may be denoted as cell-free, the sample may contain a small number of cells or cell debris while still being considered cell-free. For example, these samples may include less than or equal to about 50 cells or fewer per milliliter of sample, 45 cells per milliliter, 40 cells per milliliter, 35 cells per milliliter, 30 cells per milliliter, 25 cells per milliliter, 20 cells per milliliter, 15 cells per milliliter, 10 cells per milliliter, 5 cells per milliliter, 1 cell per milliliter, or less. In some embodiments, these samples may include greater than or equal to about 1 cell per milliliter, 5 cells per milliliter, 10 cells per milliliter, 15 cells per milliliter, 20 cells per milliliter, 25 cells per milliliter, 30 cells per milliliter, 35 cells per milliliter, 40 cells per milliliter, 45 cells per milliliter, 45 cells per milliliter, 50 cells per milliliter, or more. Such cell-free samples may include blood (e.g., whole blood), serum, plasma, saliva, urine, or mucous, for example.

### III. DEFINITIONS

As used herein, the term “amino acid” in general refers to organic compounds that contain at least one amino group, —NH₂ which may be present in its ionized form, —NH₃+, and one carboxyl group, —COOH, which may be present in its ionized form, —COO⁻, where the carboxylic acids are deprotonated at neutral pH, having the basic formula of NH₂CHRCOOH. An amino acid and thus a peptide has an N (amino)-terminal residue region and a C (carboxy)-terminal residue region. Types of amino acids include at least 20 that are considered “natural” as they comprise the majority of biological proteins in mammals and include amino acid such as lysine, cysteine, tyrosine, threonine, etc. Amino acids may also be grouped based upon their side chains such as those with a carboxylic acid groups (at neutral pH), including aspartic acid or aspartate (Asp; D) and glutamic acid or glutamate (Glu; E); and basic amino acids (at neutral pH), including lysine (Lys; L), arginine (Arg; N), and histidine (His; H).

As used herein, the term “terminal” is referred to as singular terminus and plural termini.

As used herein, the term “side chains” or “R” refers to unique structures attached to the alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−) and glutamate (−), amino acids can also be basic, such as lysine, or acidic, such as glutamic acid; uncharged polar side chains have hydroxyl, amide, or thiol groups, such as cysteine having a chemically reactive side chain, i.e. a thiol group that can form bonds with another cysteine, serine (Ser) and threonine (Thr), that have hydroxylic R side chains of different sizes; asparagine (Asn), glutamine (Gln), and tyrosine (Tyr); Non-polar hydrophobic amino acid side chains include the amino acid glycine; alanine, valine, leucine, and isoleucine having aliphatic hydrocarbon side chains ranging in size from a methyl group for alanine to isomeric butyl groups for leucine and isoleucine; methionine (Met) has a thiol ether side chain, proline (Pro) has a cyclic pyrrolidine side group. Phenylalanine (with its phenyl moiety) (Phe) and typtophan (Trp) (with its indole group) contain aromatic side groups, which are characterized by bulk as well as nonpolarity.

Amino acids can also be referred to by a name or 3-letter code or 1-letter code, for example, Cysteine; Cys; C, Lysine; Lys; K, Tryptophan; Trp; W, respectively.

Amino acids may be classified as nutritionally essential or nonessential, with the caveat that nonessential vs. essential may vary from organism to organism or vary during different developmental stages. Nonessential or conditional amino acids for a particular organism is one that is synthesized adequately in the body, typically in a pathway using enzymes encoded by several genes, as substrates allow for protein synthesis. Essential amino acids are amino acids that the organism is not unable to produce or not able to produce enough naturally, via de novo pathways, for example lysine in humans. Humans obtain essential amino acids through their diet, including synthetic supplements, meat, plants and other organisms.

“Unnatural” amino acids are those not naturally encoded or found in the genetic code nor produced via de novo pathways in mammals and plants. They can be synthesized by adding side chains not normally found or rarely found on amino acids in nature.

As used herein, β amino acids, which have their amino group bonded to the β carbon rather than the α carbon as in the 20 standard biological amino acids, are unnatural amino acids. A common naturally occurring β amino acid is β-alanine.

As used herein, the term the terms “amino acid sequence”, “peptide”, “peptide sequence”, “polypeptide”, and “polypeptide sequence” are used interchangeably herein to refer to at least two amino acids or amino acid analogs that are covalently linked by a peptide (amide) bond or an analog of a peptide bond. The term peptide includes oligomers and polymers of amino acids or amino acid analogs. The term peptide also includes molecules that are commonly referred to as peptides, which generally contain from about two (2) to about twenty (20) amino acids. The term peptide also includes molecules that are commonly referred to as polypeptides, which generally contain from about twenty (20) to about fifty amino acids (50). The term peptide also includes molecules that are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide that is produced by artificially in vitro.

As used herein, the term “subset” refers to the N-terminal amino acid residue of an individual peptide molecule. A “subset” of individual peptide molecules with an N-terminal lysine residue is distinguished from a “subset” of individual peptide molecules with an N-terminal residue that is not lysine.

As used herein the term “substituted” may refer to a compound in which one or more hydrogen atoms on the parent molecule has been replaced with another group such that the group does not substantially alter the essential function for which the compound. More specifically, the term “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, a substituent may be L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each RS is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above. A non-limiting list of possible chemical groups includes —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

As used herein, the term “fluorescence” refers to the emission of visible light by a substance that has absorbed light of a different wavelength. In some embodiments, fluorescence provides a non-destructive way of tracking, analyzing, or a combination of tracking and analyzing biological molecules based on the fluorescent emission at a specific wavelength. Proteins (including antibodies), peptides, nucleic acid, oligonucleotides (including single stranded and double stranded primers) may be “labeled” with a variety of extrinsic fluorescent molecules referred to as fluorophores.

As used herein, sequencing of peptides “at the single molecule level” refers to amino acid sequence information obtained from individual (i.e. single) peptide molecules in a mixture of diverse peptide molecules. The present disclosure may not be limited to methods where the amino acid sequence information obtained from an individual peptide molecule is the complete or contiguous amino acid sequence of an individual peptide molecule. In some embodiment, it is sufficient that partial amino acid sequence information is obtained, allowing for identification of the peptide or protein. Partial amino acid sequence information, including for example the pattern of a specific amino acid residue (i.e. lysine) within individual peptide molecules, may be sufficient to uniquely identify an individual peptide molecule. For example, a pattern of amino acids such as X-X-X-Lys-X-X-X-X-Lys-X-Lys, which indicates the distribution of lysine molecules within an individual peptide molecule, may be searched against a specific proteome of a given organism to identify the individual peptide molecule. It is not intended that sequencing of peptides at the single molecule level be limited to identifying the pattern of lysine residues in an individual peptide molecule; sequence information for any amino acid residue (including multiple amino acid residues) may be used to identify individual peptide molecules in a mixture of diverse peptide molecules.

As used herein, “single molecule resolution” refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). In one embodiment, this may include the ability to simultaneously record the fluorescent intensity of multiple individual (i.e. single) peptide molecules distributed across the glass surface. There are numerous optical devices that can be applied in this manner. For example, a conventional microscope equipped with total internal reflection illumination and an intensified charge-couple device (CCD) detector is available (see Braslaysky et al., 2003). Imaging with a high sensitivity CCD camera allows the instrument to simultaneously record the fluorescent intensity of multiple individual (i.e. single) peptide molecules distributed across a surface. In one embodiment, image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

Attribution probability mass function—for a given fluorosequence, the posterior probability mass function of its source proteins, i.e. the set of probabilities P(pᵢ/fᵢ) of each source protein pᵢ, given an observed fluorosequence fᵢ.

### III. EXAMPLES

The following examples are included to demonstrate certain embodiments of the disclosure. The techniques disclosed in the examples which follow represent techniques discovered by the inventor to function well in the practice of the disclosure. However, in light of the present disclosure, many changes can be made in the specific embodiments which are disclosed to still obtain a like or similar result without departing from the spirit and scope of the disclosure.

### Example 1—Mapping the Positions of Post-Translational Phosphorylation on Proteins at Single Molecule Sensitivity

Materials and Methods

Labeling protocol for phosphorylation peptide synthesis and purification—All peptides were synthesized with standard Fmoc chemistry using an automated solid-phase peptide synthesizer (Liberty Blue microwave peptide synthesizer; CEM Corporation). The standard Fmoc-amino acid building blocks and the Fmoc-O-benzylphosphoserine (Cat #: 03734) were purchased from Chemlmpex Inc (IL, USA). The peptides were cleaved and de-protected using acid cleavage cocktail, comprising TFA:water:triisopropylsilane (9.5:0.25:0.25 v:v:v mixture). After removal of TFA by drying with nitrogen, the peptide was precipitated with cold ether and centrifuged for 10 mins at 8000 rcf. The pellet was resuspended in acetonitrile/water (1:1 v:v mixture) and purified by high-performance liquid chromatography (Shimadzu Inc.) with an Agilent® Zorbax® column (4.6×250 mm) operating at 10 mL/min flow rate with a gradient of 5-95% methanol (0.1% formic acid) over 90 minutes. The fraction containing the peptide was collected, and the volume reduced using a rotary evaporator before lyophilization.

Synthesis of Dye-thiol reagent—3 mg of Atto 647N—NHS (Cat #: AD647N35; Atto-tec) was mixed with 150 μL basic cysteamine solution (5.1 mg cysteamine and 7.5 μL DIPEA in 1500 μL dry DMF). The mixture was incubated for 3 h and the Atto647N-S-S-Atto647N product was confirmed by mass spectrometry (Scheme 1). The product was aliquoted into glass vials, each containing 200 μg of the reagent. Single dye-thiol reagent Atto647N-SH was prepared by reacting the Atto647N-S-S-Atto647N reagent with 1 mM tris(2-carboxyethyl)phosphine (TCEP) and incubating it for 1 h at 60° C.

Labeling phosphate groups with dye-thiol reagent—Phosphorylated peptide was solubilized in 100 μL mixture of acetonitrile and water (1:1 v:v). To this solution, 46 μL of saturated barium hydroxide and 4 μL of 4M sodium hydroxide was added and incubated for 3 h at room temperature. 100 μL of DMF, 100 μL of water and 1.4 mg of TCEP was then added to the peptide solution. The entire mixture was transferred to the 200 μg of the dye-thiol reagent and incubated overnight. The TCEP addition to break the disulfide linkage in the dye-thiol reagent can be performed prior to the addition of the dye-thiol reagent to the mixture. The entire contents of the reaction was then diluted to 2 mL with acetonitrile/water mixture (1:1 v:v), and HPLC separated (as above). The fluorescent fractions, monitored at 640 nm absorbance by the diode-array detector on HPLC, were then collected, as they correspond to the phosphorylated peptide. Two signature peaks present at retention time of 54 and 55 mins, and corresponds to the unreacted dye-thiol reagent, were not collected. Following HPLC purification, labeled phosphorylated peptide was lyophilized. The N-termini of the peptides were protected by tert-Butyloxycarbonyl (“Boc”) protecting group by solubilizing the labeled peptide in DMF and incubating the mixture with tert-Butyl N-succinimidyl carbonate overnight. The solution was diluted and aliquoted into 200 μg or 2 mM.

Detection of labeled peptides—Labeled peptides were detected as in Swaminathan et al., 2010; U.S. Pat. No. 9,625,469; U.S. patent application Ser. No. 15/461,034; U.S. patent application Ser. No. 15/150,962 with minor modification. These minor modifications are: (a) The peptides were immobilized on the solid substrate via the peptide's carboxyl-terminal to an amine functionalized glass slides. (b) Prior to the experimental cycle, the “Boc” group protecting the amine termini of the peptide was de-protected by incubating the immobilized peptides with 90% Trifluoroacetic acid for 5 h at 40° C. (c) 1 mM of Trolox (6-Hydroxy-2,5,7,8-tetramethylchroman-2-carboxylic acid) dissolved in methanol was used as the imaging buffer.

Additional Labeling Strategies for Pan Phosphorylation Labeling

The phosphate group present on any modified amino acids (Serine, Threonine, Tyrosine, Histidine) can be labeled by the EDC/Imidazole reaction mechanism (shown in Scheme 1). The reaction has been described for oligonucleotides and can also be used for labeling pyrophosphates on amino acids as well and has been adapted from Wang et al., 1993. The phosphorylated peptide is reacted with 0.1 M imidazole, 0.1 M EDC and 0.25 M of donor amine (fluorophore) in pH 7.5 buffer such as PBS buffer (e.g., <10 mM). The reaction is kept at 50° C. for 20 minutes. The labeled peptide is subsequently purified and sequenced by single molecule sequencing method.

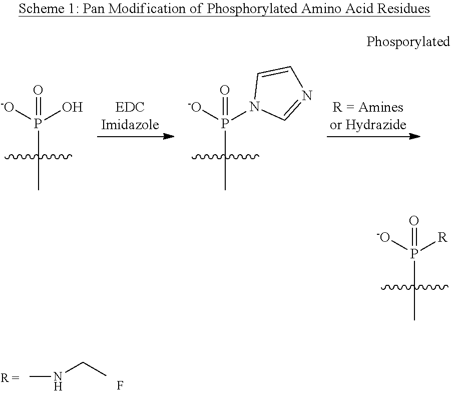

Results and Discussion

Beta elimination and Michael addition of a fluorophore via thiol conjugation has been described to fluorescently label phosphorylated peptides (Stevens et al., 2005; U.S. Pat. No. 7,476,656). However, a suitable thiol dye reagent for use in fluorosequencing, such as the Atto647N-thiol dye reagent, which contains both a sequencing suitable dye and an appropriate functional group handle, is not readily accessible. Therefore, Atto647N—S-S-Atto647N was synthesized by reaction of Atto647N—NHS with cysteamine (Scheme 2). This reaction was carried out in non-reducing and anhydrous conditions, as the presence of water can hydrolyze the NHS dye and lead to significant reduction in the reaction yield.

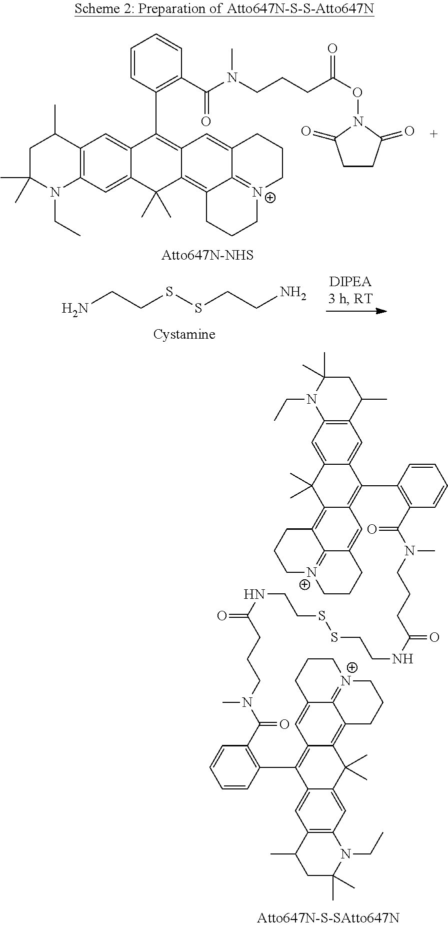 [SMILES: CCN1c2cc3c(cc2C(C)CC1(C)C)C(c1ccccc1C(=O)N(C)CCCC(=O)ON1C(=O)CCC1=O)=c1cc2c4c(c1C3(C)C)CCC[N+]=4CCC2 ; NCCSSCCN ; CCN1c2cc3c(cc2C(C)CC1(C)C)C(c1ccccc1C(=O)N(C)CCCC(=O)NCCSSCCNC(=O)CCCN(C)C(=O)c1ccccc1C1=c2cc4c5c(c2C(C)(C)c2cc6c(cc21)C(C)CC(C)(C)N6CC)CCC[N+]=5CCC4)=c1cc2c4c(c1C3(C)C)CCC[N+]=4CCC2]

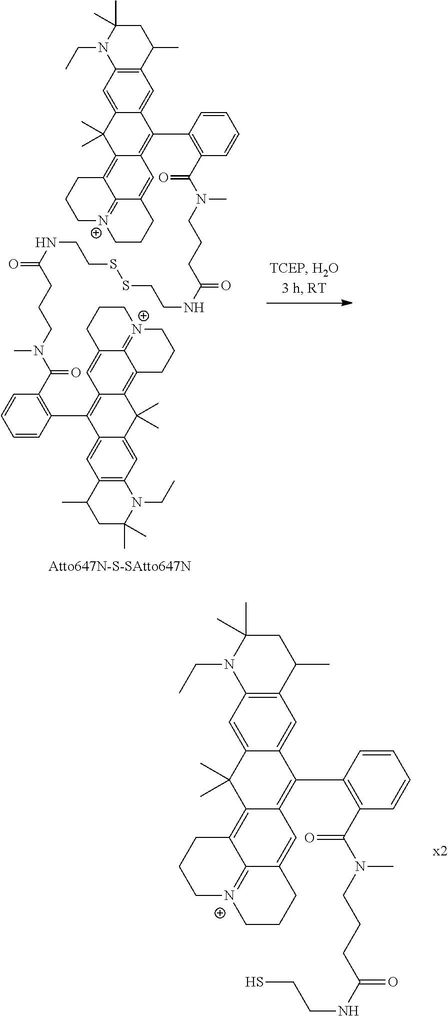 [SMILES: CCN1c2cc3c(cc2C(C)CC1(C)C)C(c1ccccc1C(=O)N(C)CCCC(=O)NCCSSCCNC(=O)CCCN(C)C(=O)c1ccccc1C1=c2cc4c5c(c2C(C)(C)c2cc6c(cc21)C(C)CC(C)(C)N6CC)CCC[N+]=5CCC4)=c1cc2c4c(c1C3(C)C)CCC[N+]=4CCC2 ; CCN1c2cc3c(cc2C(C)CC1(C)C)C(c1ccccc1C(=O)N(C)CCCC(=O)NCCS)=c1cc2c4c(c1C3(C)C)CCC[N+]=4CCC2]

To verify and optimize the labelling and fluorosequencing procedure, three phosphorylated variants of a heptad peptide were synthesized: YpSPTSPS, YSPTpSPS, and YpSPTpSPS, where pS is a phosphoserine. These heptads were then labeled by beta elimination followed by Michael addition, to fluorescently and covalently label phosphorylated serine residues with the Atto647N-thiol dye (see Scheme 3).

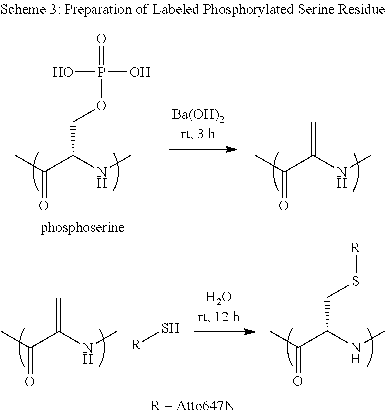

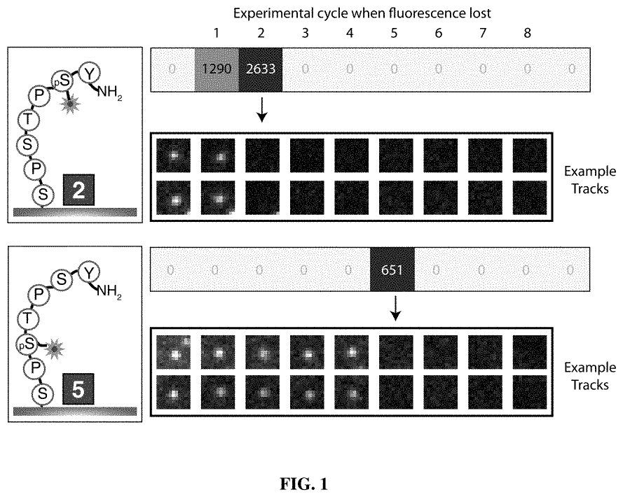

The labeled heptads were then purified by HPLC and immobilized on an aminosilane glass surface for sequencing by fluorosequencing as described in Swaminathan, 2010; U.S. Pat. No. 9,625,469; U.S. patent application Ser. No. 15/461,034; U.S. patent application Ser. No. 15/150,962; each incorporated herein by reference. As described, the fluorosequencing for a uniform population of peptides can be best described by a frequency histogram. By imaging and aligning individual peptide molecules following an Edman degradation cycle, the counts of the peptide molecules that have lost their fluorescence after the Edman cycle can be obtained. Then, by tallying the counts of peptides which lost fluorescence as a function of the Edman cycle, a frequency histogram can be obtained. By subtracting the background counts, which occur due to photobleaching and dye-losses, the counts for the significant loss events can be represented (FIG. 1). As is evident from FIG. 1, there are reductions in peptide fluorescence after the 2^{nd} Edman cycle, corresponding to the phosphoserine in the 2^{nd} position of the peptide, and after the 5^{th} Edman cycle, corresponding to the phosphoserine at the 5^{th} position. These results indicate that thiol conjugation of a fluorescent label, and subsequent additional fluorosequencing cycles, can be used map the positions of post-translational phosphorylation modifications on proteins.

An example of the method used for identifying phosphorylated residues of proteins extracted from cells is described herein. Human Embryonic kidney 293 transgenic (HEK-293T) cells were cultured and lysed using a modified RIPA buffer. Proteins were quantified and isolated from the cell lysate prior to labeling. Proteins were then denatured, and digested with the protease trypsin at a 1:50 ratio of trypsin enzyme to protein. Following digestion, a 10 kDa filter was used to filter out peptides. All phosphorylated serines and threonines in solution were then labeled using the following techniques. Phosphorylated residues were converted to the beta-eliminated variants using Ba(OH)2. A Michael addition reaction was then used to couple the fluorophore Atto 647N with a thiol modification to the beta-eliminated resides. Fluorescently labeled peptides were then purified and lyophilized.

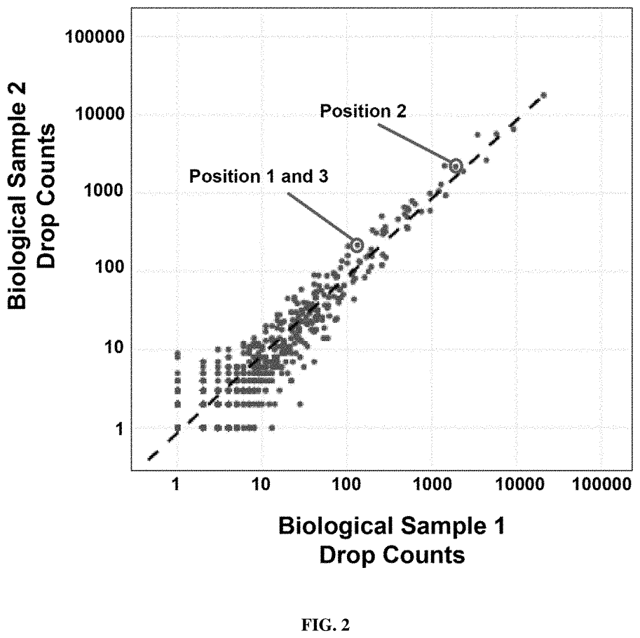

Purified peptide samples were coupled onto an amine functionalized slide surface and sequenced on the fluorosequencing platform. Counts of fluorescent drops across all amino acid positions were taken for the sequenced sample. This experiment was repeated with a different biological sample of the same cell type (HEK-293T) which was prepared and sequenced in an identical manner, serving as a source of biological replicate. These samples were sequenced and the counts of fluorescent drops across all amino acid positions were obtained. The counts from the first biological sample and the second biological sample were then plotted against each other to make the plot shown in FIG. 2. Consistent patterns denote the multiple phosphorylated residues on proteins obtained from the cell and can serve as a profile of a cell's phosphorylation status. The quantitative nature of the results spanning four orders of magnitude suggests the use for quantitative phosphoproteomics.

### Example 2—Mapping the Positions of Post-Translational Glycosylation on Proteins at Single Molecule Sensitivity

Materials and Methods

Synthesis of 1,3-dithiol modified fluorophore—Lipoic acid was reacted with tert-butyl (2-aminoethyl)carbamate using N,N′-dicyclohexylcarbodiimide (Scheme 4). The Boc protecting group was then removed by dissolving the sample in trifluoroacetic acid (TFA) and precipitating with diethyl ether. The product of this reaction, 5-[1,2]dithiolan-3-yl-pentanoic acid (2-amino-ethyl)-amide was then purified by HPLC (as above).

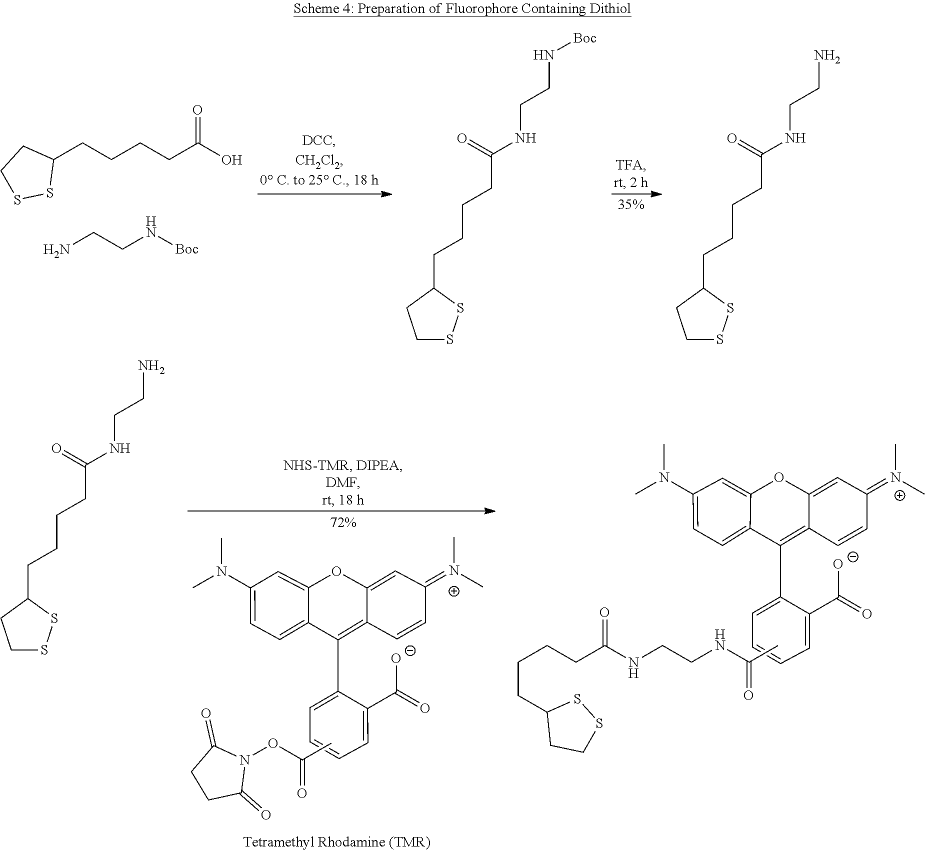

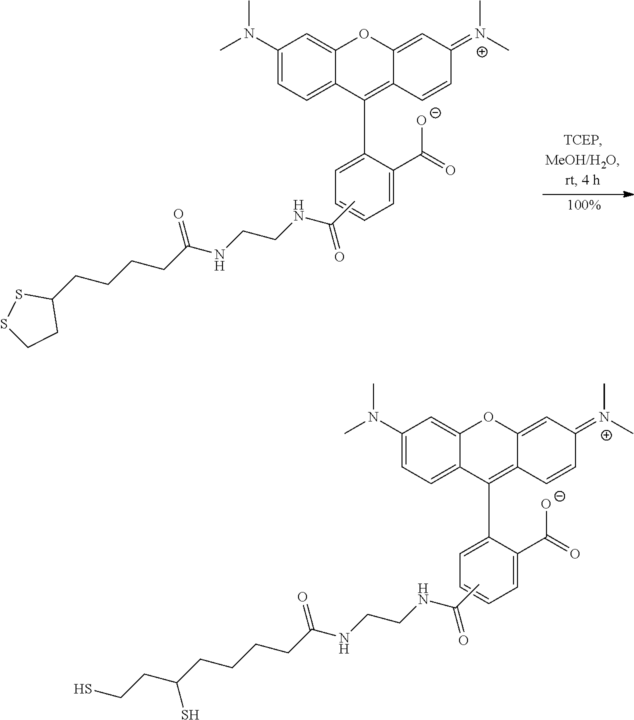

The 5-[1,2]dithiolan-3-yl-pentanoic acid (2-amino-ethyl)-amide product was then coupled with NHS activated tetramethylrhodamine (TMR) by dissolving 9.5 mg of 5-[1,2]dithiolan-3-yl-pentanoic acid (2-amino-ethyl)-amide with 10 mg of the NHS-TMR dissolved in 400 μL of an 8 mM solution of DIPEA in dimethylformamide and shaking overnight (Scheme 3). The product of this reaction was purified by HPLC (as above this 1,2-dithiolane product then had the dithiolane group reduced to 1,3-dithiol using tris(2-carboxyethyl)phosphine (TCEP) in order to form the reactive moiety for coupling to aldehydes (Scheme 3).

Conversion of 1,2-diols in sugars to aldehydes—N-acetyl-D-glucosamine will be treated with sodium periodate (Scheme 5) and the cleavage of the 1,2-diols will be verified with LCMS and NMR. Glycosylated peptides will be treated identically, to cleave the 1,2-diol groups and prepare the glycosylated peptides for fluorophore binding.

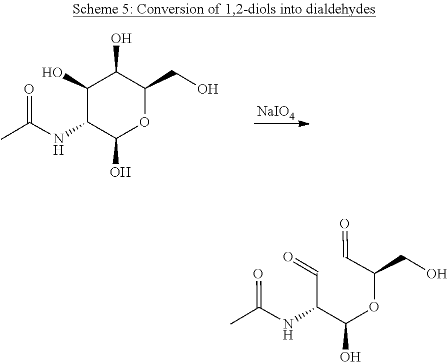

Results and Discussion

Fluorosequencing allows for low abundance variations of protein/peptide molecules to be identified and is described in Swaminathan, 2010; U.S. Pat. No. 9,625,469; U.S. patent application Ser. No. 15/461,034; U.S. patent application Ser. No. 15/150,962. This method relies on specific labeling of amino acids with fluorophores to determine its position in the peptide chain. This method can be similarly extended to identify the positions of modified amino acids by use of sugar specific fluorophores.

The concept for labeling glyocosylated amino acids is a two-step process. The first step oxidizes the alcohol groups of sugar moieties to aldehydes. The second step then reacts the dithiol reagent with the aldehyde group of the sugar molecule. It has been shown that 1,3-dithiane does not degrade when exposed to sequencing conditions, thus the inventors identified ways to modify fluorophores to have a 1,3-dithiol tether to label glycosylated amino acids.

Preparation of 1,3-dithiol tethered fluorophore—Lipoic acid was determined to be an excellent candidate for the coupling chemistry as it has a protected 1,2-dithiolane at one terminus, and a carboxylic acid on the other. The lipoic acid and NHS activated tetramethylrhodamine (TMR) were reacted according to Scheme 4, in order to generate a 1,3-dithiol modified fluorophore. This 1,3-dithiol modified fluorophore (Scheme 4, compound 10) is ready to react with glycosylated peptides to form the Edman stable 1,3-dithiane. It is important to note that this method may be used to link any NHS activated fluorophore, such as Atto657N or others, to a 1,3-dithiol tether.

Conversion of 1,2-diols in sugars to aldehydes—To confirm the viability of using sodium periodate to oxidatively cleave 1,2-diols to aldehydes while preserving the rest of the sugar structure, N-acetyl-D-glucosamine was selected. N-acetyl-D-glucosamine will be treated with sodium periodate (Scheme 5) and the cleavage of the 1,2-diols will be verified with LCMS and NMR. Interestingly, the 1,2-diol on the ring of N-Acetyl-D-glucosamine will produce two aldehydes covalently bound to each other (Scheme 5). This increases the opportunity to attach the fluorophore to the oxidized species, and may potentially lead to two fluorophores being attached at the same position of the peptide, thus increasing the brightness in scope and potentially aiding in the fluorosequencing of glycopeptides.

Fluorosequencing determination of glycosylated amino acids—It is thought that this scheme of oxidatively cleaving the 1,2-diols may then be applied to glycoproteins and glycopeptides to provide a substrate for fluorophore binding. Following fluorophore binding, these bound glycoproteins or glycopeptides can be sequenced by fluorosequencing. Fluorosequencing may be performed as above, in order to determine the location of the labeled glycosylated residue(s). This labelling and sequencing scheme is invariant to the type of glycosidic linkages, and provides a de novo method for determining the positions of the glycosylated residues on known protein or peptides.

### Example 3—Mapping the Positions of Post-Translational Lysine Trimethylation at Single Molecule Sensitivity

Materials and Methods

Synthesis of Dye-thiol reagent—As prepared for detection of post-translational phosphorylation, 3 mg of Atto 647N—NHS (Cat #: AD647N35; Atto-tec) was mixed with 150 μL basic cysteamine solution (5.1 mg cysteamine and 7.5 μL DIPEA in 1500 μL dry DMF). The mixture was incubated for 3 h and the Atto647N-S-S-Atto647N product was confirmed by mass spectrometry (FIG. 1). The product was aliquoted into glass vials, each containing 200 μg of the reagent. Single dye-thiol reagent Atto647N-SH was prepared by reacting the Atto647N-S-S-Atto647N reagent with 1 mM tris(2-carboxyethyl)phosphine (TCEP) and incubating it for 1 h at 60° C.

Hofmann elimination and reaction of peptides with fluorophore—Adapting the techniques used in the Hofmann elimination reaction, and from Brown et al., 1997, the peptides will be treated with heat and silver oxide or DIPEA in order to generate an alkene at trimethylated lysine residues (Scheme 6). These alkene containing peptides can then be reacted with a thiol-linked fluorophore such as Atto647N-SH as described above to generate peptides labeled with a fluorophore at sites of lysine trimethylation.

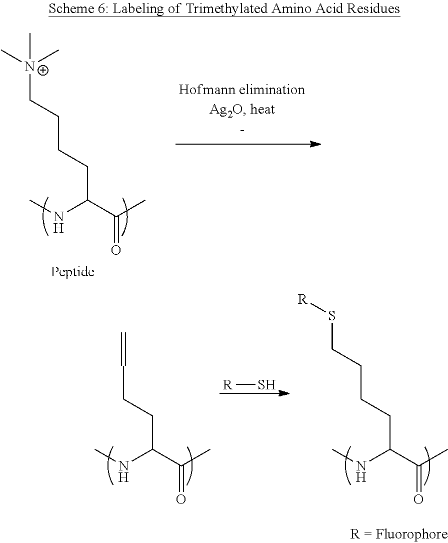

Expected Results

Fluorosequencing has been shown to precisely map the positions of fluorescently labeled amino acid residues on peptides at a sensitivity of a single molecule, and may be useful for the identification of lysine trimethylation as described in Swaminathan, 2010; U.S. Pat. No. 9,625,469; U.S. patent application Ser. No. 15/461,034; U.S. patent application Ser. No. 15/150,962. The specific attachment of a fluorophore to the trimethylated lysine residues would extend the fluorosequencing technology to map the trimethylation marks on the histone proteins, thereby aiding in the identification of the histone code.

Hofmann elimination chemistry may be used to modify the trimethylated lysine residue to a reactive alkene group, which would allow for efficient labeling with a fluorophore containing a thiol group as described above. The labeled peptides may then be sequenced by the fluorosequencing method to obtain the positions of the trimethylated lysines at single molecule resolution.

### Example 4—Mapping the Positions of Post-Translational Nitrosylation at Single Molecule Sensitivity

Nitric oxide (NO) is a cell-signaling molecule that is synthesized by a family of enzymes known as nitric oxide synthetases. NO can react with metalloproteins or covalently modify tyrosine and cysteine residues through oxidation or production of reactive nitrogen species. Nitrosylation is this category of post-translational modification that produce a covalent addition of S-nitrosylation on cysteines or nitration on tyrosine residues (See Scheme 7). Detecting and quantifying the modification have implications for better understanding of the signaling processes during stress or inflammation and developing diagnostics (Abello et al., 2009). The use of peptide mass-spectrometry for identifying the sites of nitrosylation is challenging due to—(a) unstable nature of the nitro groups and (b) the extremely low abundant modification (estimated 1 in 10⁶ tyrosine residues) (Zhan et al., 2015). Thus, single molecule fluorosequencing method would provide the ideal solution to detecting and quantifying low levels of nitrosylation modifications on tyrosines or cysteines.

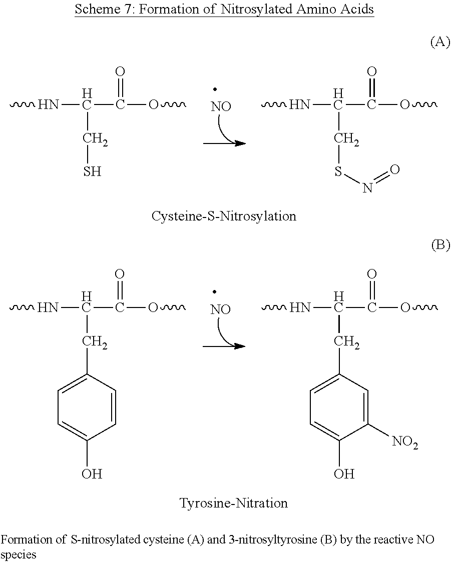

Similar to the principles used for quantifying sites of other post-translational modifications by fluorosequencing, the labeling reactions specifically targeting the nitrosyl modifications has been developed. The strategies for targeting the two different types of nitrosyl modifications are described below.

A. Cysteine—S-Nitrosylation

Bioorthogonal labeling of SNO modification has been demonstrated by organophosphine based reactions (Devarie-Baez et al., 2013) with a one-step disulfide formation. Using the same reaction principle, a one-step reaction of covalently attaching a fluorophore (reagent 2B) to the S-nitrosylated cysteine residue proposed in Scheme 7. The class of reagent comprises the organophosphine group with terminal handles (alkyne, azides) or fluorophore reagent. A two-step reaction, first with a non-fluorescing reagent followed by a fluorophore reaction to the terminal handle would produce S-nitrosyl specific fluorophore conjugate addition. A general overview of the techniques involved in modifying these amino acids are:
1. Protein/peptide isolation: Proteins are harvested from the cells using protocols common in molecular biology (Lee, 2017) and digested into peptides by common proteases, such as trypsin or GluC. In some scenarios it is feasible to fix cells by treating it with cold methanol (−20° C.) or other methods of cell fixation. Following fixation, the cells may be directly reacted with the reagent to label surface accessible PTM.
2. Blocking free thiols: In order to carry out the S-nitrosylation labeling reaction, the free thiols present on cysteine should be blocked. Two common reagents used in the procedure are iodoacetamide and N-methylmaleimide. 2-20 mM of the reagent is used at pH 7.5 buffer in order to block thiols on the peptides.
3. Labeling the SNO group: Up to 3 mM of reagent (with or without fluorophore) is incubated with the peptides or fixed cells for from about 30 mins to about 2 hours at room temperature. The excess reagent is separated by rinsing/HPLC separation or other methods such as dialysis.
4. Fluorosequencing: Fluorosequencing is performed on the fluorescently labeled peptides.

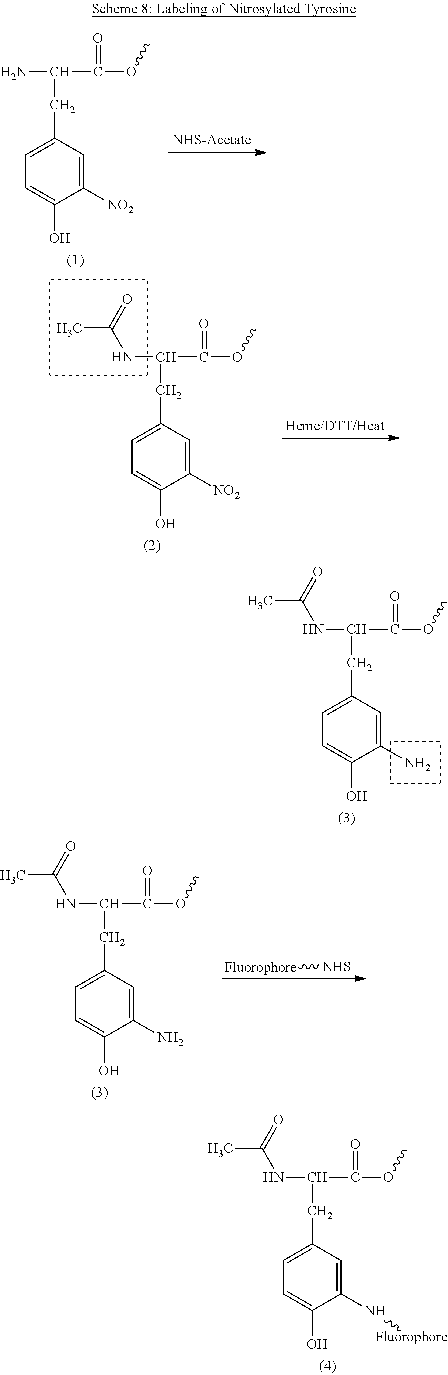

Schematic of the techniques for labeling 3-nitrotyrosine residue in peptides or proteins with fluorophore. The (1) nitrated tyrosine (shown in this example as the N-terminal residue) is reacted with NHS-acetate that acetylates all the free amines present on the peptide (2). Addition of Heme/DTT under boiling conditions converts the nitro group into an amine moiety (3). This amine group reacts with fluorophore—succinimidyl ester to covalently label the 3-nitrotyrosine residue (4). The fluorescently labeled peptide can now be subjected to fluorosequencing for analysis.

This method can thus localize the residues of modification and quantify the stoichiometry of PTM labeling of the cysteine residue. Other variants of ligation of fluorophore with the intermediate phosphine adduct can be performed such as dehydroalanine formation as indicated in literature (Devarie-Baez et al., 2013).

B. Tyrosine Nitration:

The common chemical derivatization strategy for nitrotyrosine, used in mass-spectrometry proteomics is a two-step process. The first step is the reduction of the nitro group to the amino group followed by covalently labeling the amino group with a specialized reagent. Prior to this step, the other amino groups on the peptides/proteins are blocked, typically by acetylation (Abello et al., 2010; Devarie-Baez et al., 2013). This strategy (See Scheme 8) can be directly adapted for labeling the nitrotyrosine group with a distinct fluorophore for fluorosequencing. A method for labeling the nitrotyrosine for fluorosequencing application is described as follows:
1. Protein/peptide isolation: The isolated proteins and peptides are solubilized in sodium phosphate buffer (pH 7.5). The digested proteins or peptides can be lyophilized prior to analysis. The approximate concentration of the peptide is 10 μM.
2. Acetylation of amines: All the free amines and other nucleophiles are acetylated by incubating 190 μL of the nitrated peptide with NHS-Acetate (final concentration of 25 mM) for 2 h at room temperature. The O-acetylations were reversed and excess reagent hydrolyzed by boiling the reaction for 15 minutes.
3. Reduction of nitrotyrosine to aminotyrosine: DTT (final concentration: 20 mM) and Hemin (25 μM) was added to the sample and incubated for 15 minutes in a boiling water bath.
4. Fluorescent labeling: Atto-NHS or other fluorophore-NHS (2 mM) was added to the solution and incubated for 2 h at room temperature. Excess dyes were removed by HPLC or other separation method prior to fluorosequencing.

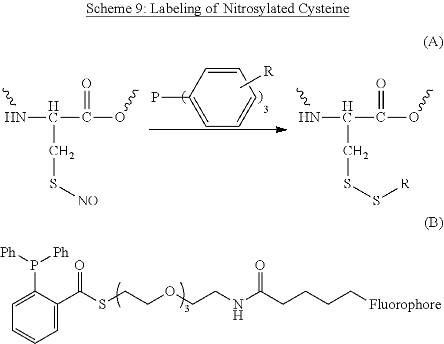

Schematic of the one-pot reaction for selective labeling of S-nitrosylated cysteine. (A) After alkylating the free thiols, the use of an organophosphine reagent yields a disulfide linkage. (B) A generic example of a reagent with a fluorophore connected to the phosphine group is provided.

The one-pot process described in the above section is uniquely suited for localizing and quantifying the nitrotyrosine positions on peptides and proteins.

### Example 5—Mapping the Positions of Post-Translational Citrullination at Single Molecule Sensitivity

Citrullination is a post-translational modification caused by enzyme Protein Arginine deiminase (PAD) where the arginine side chain is converted to citrulline (process called deimination). The conversion leads to a change in the mass by 1 Da, the loss of the positive charge and two potential hydrogen bond donors. The modification has a major effect on protein structure and stability and is implicated in autoimmune disorders, neurodegenerative diseases and in tumor biology (Gyorgy et al., 2006). The small mass change overlaps with the isotopic distribution of unmodified Arginine residues in peptide mass-spectrometry, making its identification challenging. Similar to the other questions in PTM, developing an assay for localizing and quantifying the low abundant citrullinated residue is important.

A chemoselective strategy for targeting citrullinated residue has been demonstrated. A phenylglyoxal reagent reacts with arginine (under basic) and citrulline (under acidic conditions) forming a five membered ring. Although under acidic conditions, the reagent additionally binds to homocitrulline and cysteine, the thiohemiacetal ring formed with cysteine is hydrolysed in neutral pH. A method has been described for fluorescently labeling citrullinated residues with rhodamine using the phenylglyoxal reagent (Bicker et al., 2012). This procedure would be adapted for fluorosequencing as follows (See Scheme 10):
1. Protein/peptide isolation: The isolated proteins are digested or the peptide is isolated according to standard well optimized procedures. About 50 μM of citrullinated peptides is lyophilized or solubilized in 50 mM HEPES buffer (pH 7.5)
2. Thiol group on cysteines are capped using iodoacetamide or fluorescent dyes, which prevents the cross-reactivity of the citrulline specific reagent. 2 mM iodoacetamide alkylates the thiol groups in the protein digest.
3. The citrulline containing peptide was incubated with 5 mM phenylglyoxal reagent and 20% Trichloroacetic acid (pH<1) for 3 hours at 37° C.
4. The phenylglyoxal reagent can be directly coupled with a fluorophore or contain a handle (click handle) for subsequent reaction with a fluorophore.
5. The excess reagent is purified from the labeled citrullinated peptide for fluorosequencing.

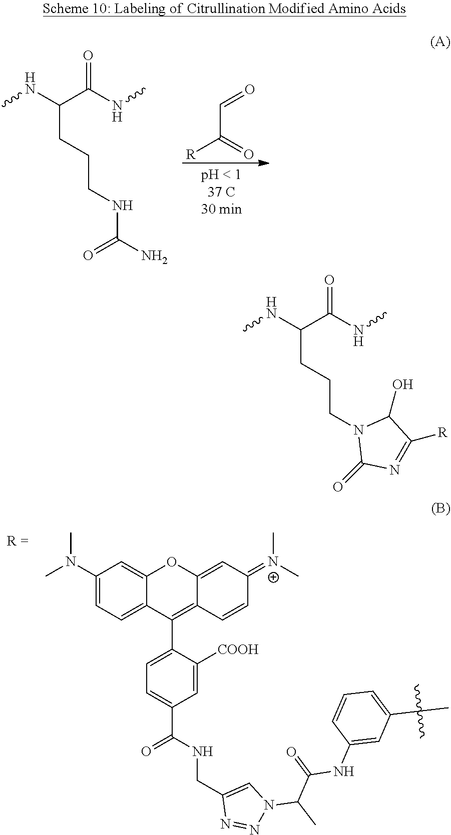

Selective labeling of citrullinated residue by Rhodamine-Phenylglyoxal reagent. (A) Reaction conditions for labeling of citrullinated residue. (B) Rhodamine—phenylglyoxal reagent used for fluorescently labeling citrullinated residues for fluorosequencing.

### Example 6—Mapping the Positions of Post-Translational Sulfenylation at Single Molecule Sensitivity

Sulfenic acid is one of a specific oxidative modification of cysteine residue which is formed upon reaction of the thiol side chain with mild oxidizing environment. The modification is a readout of early stages of reactive oxygen species formation, the intermediate step for formation of disulfide bond formation and also involved in redox signaling (Poole et al., 2004). The unstable nature of the bond under commonly used ionization conditions in mass spectrometers makes localizing and quantifying the modification extremely challenging. However, the reactive nature of the group enables chemical coupling and enrichment of the modified peptides (Poole et al., 2007; Reddie et al., 2008) feasible. The principle is the selective reaction of the sulfenic acid with dimedone (5,5-dimethyl-1,3-cyclohexanedione) which has been linked to several fluorescent reagents (See Scheme 11). Additionally, a biotin labeled reagent may be used (Millipore; Cat #NS1226-1MG).

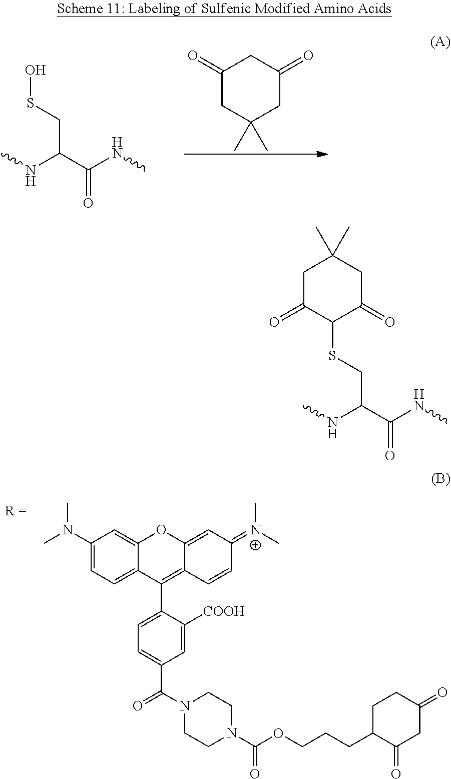

Reaction illustrating the selective labeling of sulfenic acid with 1,3-cyclohexanedione reagent derivative. (A) High yielding reaction was demonstrated by using dimedone (5,5-dimethyl-1,3-cyclohexanedione). (B) An example of Rhodamine-derivative for labeling sulfenic acid modification feasible for fluorosequencing

Below is a reaction method for labeling sulfenic acid on peptides with derivatized rhodamine for fluoro sequencing:
1. Protein/peptide isolation: The proteins were digested or the peptides were isolated using common standardized procedures. About 1-10 μmol peptides were lyophilized or solubilized in phosphate buffer (pH 7; 25 mM) and 1 mM EDTA.
2. Labeling of sulfenic acid: The fluorescent reagent was added to a concentration of 5 mM and incubated for 2 h at 37° C. The reagent can be two halves—one with an azide handle and the second with a fluorophore that specifically reacts with the linker.
3. The excess reagents and fluorophores are purified away before fluorosequencing.

There are a number of other labeling reactions involving different reagents and reaction mechanisms that have also been demonstrated (Gupta and Carroll, 2014).

### Example 7—Measurement of Post-Translational Modification as a Biomarker

As described above, the precise sites of post-translational modifications, such as phosphorylation state, affects the function of proteins and may serve as a reliable indicator of disease state. One such molecule, troponin, is a diagnostic biomarker for cardiac dysregulation (Wijnker et al., 2014). However, the site-specific nature of the phosphorylation is an important diagnostic and therapeutic marker for understanding and treating heart failures (Zhang et al., 2012). Depending on the phosphorylation state and sites on the troponin molecule, the diagnosis may range from exercise to a disease state as severe as cardiac myopathy.

The methods presented above can be easily adopted to assess the phosphorylation state of a number of potential phosphorylation related biomarkers. The first step would be to perform a standard antibody pulldown for the protein of interest, i.e. troponin. Then the enriched protein may be digested into shorter peptides using a protease, such as GluC or trypsin, producing peptides of a specific length. The phosphorylation sites can then be labelled on the peptide molecules as described in Example 1. This would allow for the exact locations of the post-translational modifications to be identified and quantified by fluorosequencing, offering significant advantages over current diagnostic tests such as semi-quantitative antibody assays like those used to measure the levels of troponin or phosphorylated troponin in a sample. This methodology may also be applied to assessing the methylation or glycosylation of any protein as well, providing new biomarkers for diseases which are characterized by post-translational modifications of the proteins.

All of the methods disclosed and claimed herein can be made and executed without undue experimentation in light of the present disclosure. While the compositions and methods have been described in terms of certain embodiments, variations may be applied to the methods and in the techniques or in the sequence of techniques of the method(s) described herein without departing from the concept, spirit and scope of the disclosure. More specifically, it will be apparent that certain agents which are both chemically and physiologically related may be substituted for the agents described herein while the same or similar results would be achieved. All such similar substitutes and modifications are deemed to be within the spirit, scope and concept of the disclosure as defined by the appended claims.

### REFERENCES

The following references, to the extent that they provide procedural or other details supplementary to those set forth herein, are specifically incorporated herein by reference.
Abello et al., Talanta Analytical Proteomics, 80:1503-1512, 2010.
Abello et al, J. Proteome Res., 8:3222-3238, 2009.
Aebersold et al., Nat Chem Biol., 14: 206-214, 2018.
Ardito et al., Int J Mol Med.; 40: 271-280, 2017. doi:10.3892/ijmm.2017.3036
Bicker et al., J. Am. Chem. Soc., 134:17015-17018, 2012.
Braslaysky et al., Proc. Natl. Acad. Sci., USA, 100(7):3960-4, 2003.
Brown et al., J. Am. Chem. Soc., 119(14): 3288-3295, 1997.
Czernik et al., Regulatory Protein Modification, Humana Press, pp. 219-250, 1997.
Devarie-Baez et al., Methods San Diego Calif, 62:171-176, 2013.
Du and Huang, Yi chuan=Hered., 29: 387-92, 2007.
Frese et al., J Proteorne Res. 12: 1520-5, 2013.
Garcia et al., Nat Methods., 4: 487-489, 2007.
Gupta and Carroll, Acta BBA—Gen. Subj., Current Methods to Study Reactive Oxygen Species—Pros and Cons, 1840, 847-875, 2014.
György et al., Int. J. Biochem. Cell Biol., 38:1662-1677, 2006.
Huang and Chang, Prostate Cancer—From Bench to Bedside, Ch. 8, 2011.
Korff et al., Heart, 92: 987-93, 2006.
Lee, Endocrinol. Metab., 32:18-22, 2017.
Mondragón-Rodriguez et al., Neuropathol Appl Neurobiol., 40(2):121-35, 2014.
Onder et al., Expert Rev Proteomics, 12: 499-517, 2015.
Poole et al., Annu. Rev. Pharmacol. Toxicol., 44:325-347, 2004.
Poole et al., Bioconjug. Chem., 18:2004-2017, 2007.
Reddie et al., Mol. Biosyst., 4:521-531, 2008.
Solari et al., Mol Biosyst., 11: 1487-93, 2015.
Stevens et al., Rapid Commun Mass Spectrom., 19: 2157-2162; 2005.
Stowell et al., Annu Rev Pathol Mech Dis. 10:473-510, 2015.
Swaminathan R, Biology S. Jagannath Swaminathan. Education. doi:10.1002/rcm.3179, 2010.
U.S. patent application Ser. No. 15/510,962.
U.S. patent application Ser. No. 15/461,034.
U.S. Pat. No. 7,476,656.
U.S. Pat. No. 9,625,469.
von Hofmann, Ann der Chemie and Pharm., 78:253-286, 1851.
Wagner and Carpenter, Nat Rev Mol Cell Biol., 13:115-126, 2012.
Wijnker et al., Neth Heart J., 22: 463-9, 2014.
Zhan et al., Mass Spectrom. Rev., 34:423-448, 2015.
Zhang et al., Circulation, 126: 1828-1837, 2012.

1.-60. (canceled)

61. A method of identifying a post translational modification on an amino acid residue of a peptide or a protein, the method comprising:
(a) contacting said peptide or said protein with a labeling reagent under conditions such that said labeling reagent interacts with said post translational modification on said amino acid residue of said peptide or said protein to covalently couple said labeling reagent or derivative thereof to said amino acid residue, thereby yielding a labeled peptide or a labeled protein; and
(b) sequencing said labeled peptide or said labeled protein.

62. The method of claim 61, wherein said post translational modification on said amino acid residue comprises phosphorylation, glycosylation, nitrosylation, citrullination, sulfenylation, or trimethylation.

63. The method of claim 61, wherein said contacting said peptide or said protein with said labeling reagent comprises reacting said peptide or said protein comprising said post translational modification with a phosphine.

64. The method of claim 61, wherein said contacting said peptide or said protein with said labeling reagent comprises reacting said peptide or said protein comprising said post translational modification with a glyoxal group.

65. The method of claim 61, wherein said sequencing comprises a fluorosequencing method.

66. The method of claim 65, wherein said fluorosequencing method comprises labeling at least one amino acid of said peptide or said protein which does not contain a post translational modification with a second labeling reagent.

67. The method of claim 65, wherein said fluorosequencing method comprises sequentially removing amino acid residues of said peptide or said protein until said amino acid comprising said post translational modification is removed.

68. The method of claim 67, where said sequentially removing said amino acid residues comprises contacting an N-terminal amino acid of said peptide with an isothiocyanate and an acid, microwave irradiation, or heat.

69. The method of claim 67, wherein said sequentially removing said amino acid residues comprises enzymatically cleaving at least a subset of said amino acid residues.

70. The method of claim 61, wherein said sequencing is at a single molecule level.

71. The method of claim 61, wherein said covalently coupling said labeling reagent or said derivative thereof to said amino acid residue forms a covalent bond between said post translational modification on said amino acid residue of said peptide or said protein and said labeling reagent.

72. The method of claim 71, wherein said labeling reagent or said derivative thereof is directly covalently bonded to said post translational modification on said amino acid residue of said peptide or said protein.

73. The method of claim 71, wherein said labeling reagent or said derivative thereof is covalently coupled through an intermediary molecule to said post translational modification on said amino acid residue of said peptide or said protein.

74. The method of claim 61, wherein said contacting said peptide or said protein with said labeling reagent comprises:
(i) reacting said peptide or said protein under conditions such that said post translational modification on said peptide or said protein is converted to a reactive group, thereby forming a reactive peptide or a reactive protein;
(ii) reacting said labeling reagent with said reactive peptide or said reactive protein to form said labeled peptide or said labeled protein.

75. The method of claim 74, wherein said post translational modification comprises phosphorylation, and wherein said reacting said peptide or said protein comprises contacting said peptide or said protein with a base.

76. The method of claim 74, wherein said post translational modification comprises phosphorylation, and wherein said reacting said peptide or said protein comprises contacting said peptide or said protein with an activating agent and a base.

77. The method of claim 74, wherein said post translational modification comprises trimethylation, and wherein said reacting said peptide or said protein comprises contacting said peptide or said protein with silver oxide (Ag₂O).

78. The method of claim 74, wherein said post translational modification comprises glycosylation, and wherein said reacting said peptide or said protein comprises contacting said peptide or said protein with an oxidizing agent.

79. The method of claim 74, wherein said post translational modification comprises nitrosylation, and wherein said reacting said peptide or said protein comprises contacting said peptide or said protein with a reducing agent.

80. The method of claim 74, wherein said post translational modification comprises nitrosylation, and wherein said reacting said peptide or said protein comprises contacting said peptide or said protein with a phosphine.
